(19) United States
(12) Patent Application Publication
(10) Pub. No.: US 2012/0321706 A1
(43) Pub. Date: Dec. 20, 2012

(54) NOVEL GASTRORETENTIVE DOSAGE FORMS OF POORLY SOLUBLE DRUGS

(75) Inventors: Suher Masri, Jerusalem (IL); Eytan Moor, Jerusalem (IL); David Kirmayer, Maale Adumim (IL); Elena Kluev, Jerusalem (IL); Giora Carni, Tel Aviv (IL); Nadav Navon, Rehovot (IL)

(73) Assignee: INTEC PHARMA LTD., Jerusalem (IL)

(21) Appl. No.: 13/502,572

(22) PCT Filed: Oct. 19, 2010
(86) PCT No.: PCT/IB2010/002867

§ 371 Date: Sep. 5, 2012

Related U.S. Application Data
(60) Provisional application No. 61/252,922, filed on Oct. 19, 2009.

(51) Int. Cl. A61K 9/48; A61K 31/565; A61K 31/58; A61K 31/551; A61K 31/495; A61K 31/545; A61K 31/7048; A61K 31/57; A61K 31/573

(52) U.S. Cl. 424/455; 424/451; 424/463; 514/177; 514/182; 514/174; 514/179; 514/255.04; 514/197; 514/27; 514/221; 424/456

(57) ABSTRACT

Disclosed is a multi-layered gastroretentive dosage form for the controlled release of a poorly soluble drug or diagnostic in the stomach and gastrointestinal tract of a patient, folded into a capsule which disintegrates rapidly and the said multi-layered dosage form unfolds rapidly upon contact with the gastric juice. The mechanisms of the gastric retention are not dependent on and do not influence the materials and methods used in controlling the release of the said poorly soluble drug.

### CROSS-REFERENCE TO RELATED APPLICATIONS

The present application claims priority to U.S. Provisional Patent Application No. 61/252,922 filed on Oct. 19, 2009, the disclosure of which is herein incorporated in its entirety.

### BACKGROUND OF THE INVENTION

The present invention relates to the field of pharmaceutics and more particularly, to a controlled-release drug delivery system for retention in the stomach for prolonged time intervals, using a suitable technology and delivery of poorly soluble drugs and diagnostics comprising a multilayered gastroretentive dosage form comprising films, layers or membranes, and pores or orifices.

The controlled-release drug delivery systems for gastric retention were extensively reviewed lately (AAPS Pharm. Sci. Tech. 2005; 6 (3) Article 47-Sh. Arora et al, Floating Drug Delivery Systems: A Review), Journal of Controlled Release, 63 (2000) 235-259, “Floating drug delivery systems: an approach to oral controlled drug delivery via gastric retention”; J. Control. Release, 2003; 90 (2): 143-62, “Expandable gastroretentive dosage forms”. Klausner E. A., Lavy E, Friedman M, Hoffman A.; Exp. Opin. Drug. Deliv. 2006; 3 (2): 217-33, “Gastroretentive drug delivery systems”. Streubel A, Siepmann J, Bodmeier R.

These systems have a remarkable advantage for the delivery of drugs which (1) have a “narrow absorption window” in the gastrointestinal tract, i.e. are preferentially absorbed in the duodenum and/or jejunum over ileum and/or colon, or have better solubility in upper parts of the gastrointestinal tract; (2) are intended for local treatment of proximal parts of the gastrointestinal tract (stomach and/or duodenum); and (3) the drugs which degrade in the colon or in the intestines.

However, a large group of drugs suffers also from poor solubility in aqueous medium. These drugs usually require additional formulative effort, and the advances in drug delivery of poorly soluble drugs have been recently summarized in the book “Water-insoluble drug formulation”, by CRC press, edited (2^{nd} edition) by Rong Liu. One of the examples given therein is itraconazole delivery with the aid of a mucoadhesive system three-layered tablet. The described delivery system also involves layering of the drug onto carrier powder and blending with regular tablets excipients.

Controlled-release (CR) drug delivery systems for the poorly soluble drugs are well known for the skilled in the art; however these systems suffer from significant disadvantages. A CR dosage form, by the definition, releases the drug in a sustained, controlled manner. This is usually aimed to improve the outcome of the therapy through reduction of dosing frequency, usually associated with reduced peak blood concentrations and the peak-associated side effects. Nevertheless, the controlled-release dosage form of a poorly soluble drug must provide the means of drug release from the controlled-release dosage form. In addition, already released poorly soluble drug is usually co-localised with the dosage form in the gastrointestinal tract, which frequently leads to super saturation of the medium with the poorly soluble drug and leads to a phenomenon of reprecipitation, whereby the dissolved poorly soluble drug precipitates, frequently as a less soluble free drug form. Reprecipitation frequently precludes further absorption, as the drug may never redissolve again.

There is a physiological mechanism that the body employs to facilitate the dissolution and absorption of the poorly soluble drugs. The association with the dietary fat and incorporation into the mixed micelles is an important pathway of absorption. The drugs may further follow the lymphatic pathway of absorption, associated with chylomicrons. The bile acids that are excreted into the duodenum act as surface-active agents assist the stabilisation of the mixed micelles, and by that solubilise the poorly soluble drugs as well. The concentration gradient is naturally formed through the intestines, with peak concentration at the opening of the bile ducts and reducing downstream as bile salts are absorbed. This causes yet another variance factor for conventional controlled-release dosage forms that are unlimited to one location in the gastrointestinal tract.

The literature recognises as well the need for separate control mechanisms for the gastric retention and for the controlled release of a drug. Most frequently however, the dosage forms that pertain to gastric retention rely on the same materials to produce the gastric retention and the controlled release. There are only few examples in the art wherein the gastric retention can be controlled through separate mechanisms with the gastric retention.

The majority of the systems aimed at retention in the stomach for prolonged time intervals are based on hydrophilic materials that swell to large dimensions. Releasing poorly soluble drugs from such matrices is a cumbersome task and is often accompanied by the matrix erosion.

As the release from such system may often be impeded by hydrophilic regions, either membranes or matrices, a dedicated pathway for releasing the poorly soluble drugs is needed.

### SUMMARY OF THE INVENTION

The present invention comprises a controlled-release dosage form that retains in the stomach for prolonged time intervals, wherein said dosage form comprises at least one drug or diagnostic having low aqueous solubility, and wherein said poorly soluble drug or diagnostic is released from the system through a dedicated pathway substantially devoid of hydrophilic materials, excluding aqueous medium of dissolution.

In another aspect, the present invention comprises a gastroretentive dosage form wherein the retention in the stomach is not dependent upon the presence or absence of said dedicated pathways and wherein the release rate of the said poorly soluble drug or diagnostic is governed solely by the materials that are not required to impart the system the properties of gastric retention.

### BRIEF DESCRIPTION OF THE DRAWINGS

FIG. 1 shows a schematic drawing of the film components of poorly soluble drug or diagnostic gastroretentive dosage form and their approximate dimensions

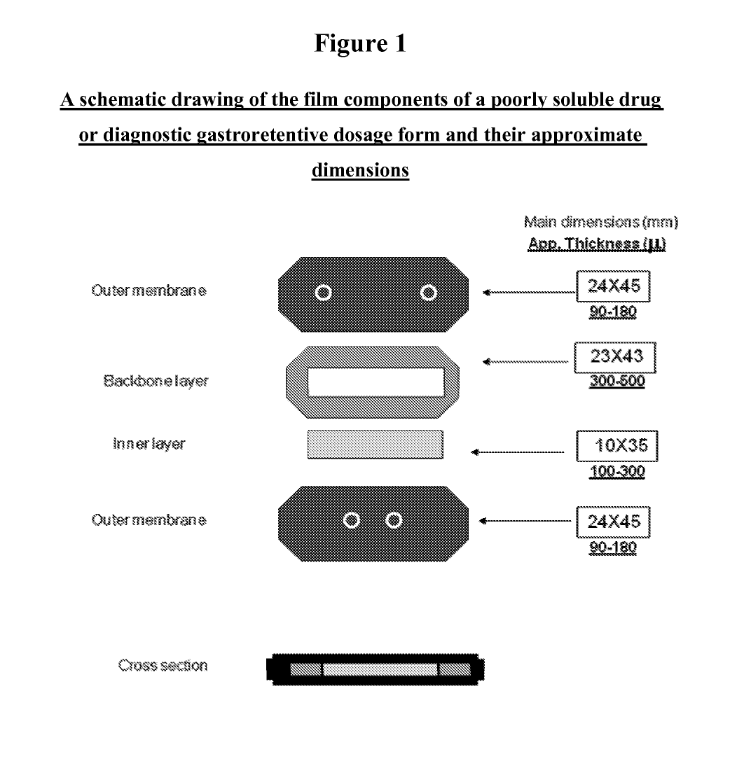

### DETAILED DESCRIPTION OF THE INVENTION

### Definitions

The wording hereinbelow is implied in the common meaning of the definitions and statements as known to the versed in the art of pharmaceuticals and polymer science. However, there are several terms that should be understood in the concept of present invention as follows:

“Gastroretentive dosage form(s)” (GRDF or GRDFs in the plural) refers to dosage forms with delayed gastric emptying or longer retention in the stomach as compared to food.

“Gastroretentive” or “gastric-retentive” dosage forms denote dosage forms comprising multilayer structures including an inner layer, a rigid frame layer and one or two outer layers, or comprising an inner layer, a rigid frame layer, one or two outer layers and one or two supra-outer layers, or comprising the foregoing structures folded or compacted into a capsule. The capsule disintegrates rapidly upon contact with gastric fluid and the structures unfold rapidly upon contact with gastric juice and reside in the stomach of a mammal, preferably a human, for prolonged periods of time, preferably longer than food and small indigestible particles of size below 10 mm in either dimension “Gastric retention” is therefore the maintenance or withholding of a drug in stomach, for a time period longer than the time it would have been retained in the stomach when delivered in a free form or within a gastro-intestinal (GI) delivery vehicle which is not considered gastroretentive. Gastroretentivity may be characterized by retention in the stomach for a period that is longer than the normal emptying time from the stomach, i.e. longer than about 2 hours, particularly longer than about 3 hours and usually more than about 4, 6, 8 or 10 hours. Gastroretentivity typically means retention in the stomach from about 3, 4, 6, 8, 10 or at times 18 hours up to about 21 hours.

“Controlled-release drug delivery system” is used herein in reference to a dosage form of a poorly soluble drug, whereas the latter is released from the said dosage form in a controlled manner over designable time intervals at needed quantities to produce a prolonged, sustained or delayed pharmacological effect that is otherwise unattainable through conventional non-modified-release dosage forms. More specifically, the term is referring to the system of present invention. It can also be generally understood as known to the versed in the art.

“Simulated gastric fluid” and “Simulated intestinal fluid” as used herein refers to solutions “Gastric fluid, Simulated, TS” and “Intestinal fluid, Simulated, TS” as it appears in the United States Pharmacopeia 30, without enzymes.

“Gastric medium” and “Intestinal medium” as used herein refer to a biological medium of the stomach and intestines respectively, or an artificial medium, used to mimic the environment of the stomach or intestines.

The term “degradable” as used herein is intended as capable of being chemically and/or physically reduced or broken down in the body of a patient and within a relevant time period.

The phrase “polymer which is not instantly soluble in gastric fluid” as used herein means that the polymer will gradually dissolve in the GI tract during its residence therein.

The term “inert” or “inactive” or “inactive ingredient” as used herein refers to components in the internal layer or compartment, outer membranes, optional layers and/or the immediate release layers that do not react with the active ingredient or affect its properties, or cause any biological effect upon administration to a subject.

The phrase “prolonged period” as used herein intends a period of delivery of at least 80% of the dose that lasts for several hours to about 24 hours, usually up to about 12 hours, and often between about 3 and 7 hours.

The terms “swellable” and “swelling” mean, with respect to a polymer, that the polymer is capable of imbibing fluid and expanding when in contact with fluid present in the environment of use.

A “patient” as referenced herein is a human or non-human mammal who may receive the gastroretentive drug formulations of the present invention.

“Treating” or “treatment”, and the like are used herein to refer to obtaining a desired pharmacological and physiological effect. The effect may be prophylactic in terms of preventing or partially preventing a disease, symptom or pathological condition and/or may be therapeutic in terms of a partial or complete cure of a disease, condition, symptom or adverse effect attributed to a pathological condition. Thus, “treatment” covers any treatment of a disease in a mammal, particularly a human, and includes: (a) preventing a pathological condition from occurring in an individual which may be predisposed to develop a pathological condition but has not yet been diagnosed as having it, i.e., causing the clinical symptoms of a pathological condition not to develop in a subject that may be predisposed to develop the condition but does not yet experience or display symptoms of the condition; (b) inhibiting, i.e., arresting or reducing the development of the pathological condition or its clinical symptoms; or (c) relieving symptoms associating with the pathological condition.

“Drug”, “active pharmaceutical ingredient”, “API”, “active agent”, “active ingredient”, “active”, and the like, are used in connection with the present invention as pure chemical substances, mixtures of pure chemical substances or crude extracts from various sources, which are used to treat pathological conditions of a person in need or such treatment. “Inactive ingredient”, “excipient”, “material”, “component”, “ingredient”, “inactive material”, “inactive”, “agent”, and the like as used interchangeably herein, refer to materials or ingredients that are not drugs, but are employed in pharmaceutical compounding in connection with the present invention with intention to impart the final dosage form specific characteristics, as known to the versed in the art, and partially described in detail hereinbelow with reference to specific characteristics imparted to the system of present invention.

“Film” or “layer” or membrane” is used interchangeably in connection with the components of the multi-layered GRDF of this invention and their formulation and preparation is described in the following.

As used in the specification and claims, the forms “a”, “an” and “the” include singular as well as plural references unless the context clearly dictates otherwise.

Further, as used herein, the term “comprising” is intended to mean that the system includes the recited elements, but not excluding others which may be optional in the design of the system, such as fillers and the like. The term “consisting essentially of” is used to define a system that includes the recited elements but exclude other elements that may have an essential significance effect on the performance of the system.

“Consisting of” shall thus mean excluding more than traces of other elements. Embodiments defined by each of these transition terms are within the scope of this invention.

Without wishing to be bound by a specific theory, we believe that the prolonged retention of a poorly soluble drug in the stomach presents several advantages. Firstly, the drug is exposed to gastric environment for prolonged time intervals. This means that the dosage form is exposed to the fresh volumes of gastric medium throughout the residence period. There is a basal level of secretions in the stomach, in addition to the induced secretory function in anticipation and in the presence of food. These secretions are naturally devoid of the drug that is released from the gastroretentive controlled-release dosage form. This makes a perfect medium to dissolve the relative small amount that is released through the same time interval. Moreover, the released drug or diagnostic is rapidly cleared from the stomach, avoiding super saturation and significantly diminishing the chance for the reprecipitation, albeit still being exposed to sharp pH changes. These changes, we believe, are less significant due to the low dose that is exposed to sharp pH difference each time. Therefore, each dose fraction of the poorly soluble drug will have the relative fraction of “dedicated” gastric and intestinal medium. Secondly, the relative fractions released from such system will benefit from “dedicated” portion of the bile salts, increasing therefore the amount of bile salts that are available per dose in proportion to the gastric residence. Thirdly, many drugs are weak bases and use acid salts to increase their solubility. The natural pH of the medium in the stomach is acidic, which prevents precipitations and assists the bioavailability of these drugs. On the other hand, the drugs that are weak acids are delivered to the more neutral pH in a sustained manner, allowing for better dissolution in the suitable environment.

In current embodiments, the present invention aims to improve the pharmacokinetic parameters of the poorly soluble drugs by using special configurations of a gastroretentive dosage form known as the “accordion pill”. The “accordion pill”, has been described in detail in previous publications, for example U.S. Pat. No. 6,685,962, PCT application WP 2007/093999 and PCT application WO07083309 which are incorporated herein by reference in their entirety.

In one embodiment, there is provided a gastroretentive dosage form which will increase the retention of a poorly soluble drug in the stomach resulting in beneficial increase of exposure to the drug, steadier blood plasma levels in comparison to the commercially available dosage forms and decreased variation between the subjects and dosing periods.

The present invention presents a controlled-release drug delivery system, capable of being retained in the stomach for prolonged time intervals, wherein said system comprises a drug or diagnostic that has a limited solubility in aqueous media.

In current embodiments, the poorly soluble drug or diagnostic in the multilayered gastroretentive dosage forms of this invention is a very slightly soluble material as defined by the United States Pharmacopeia 31, or with the aqueous solubility below this definition.

The drugs suitable for use with the GRDFs of this invention are selected from the group comprising but not limited to progesterone, tacrolimus, estradiol, budesonide, dipyridamole, norgestrel, alendronate, amlodipine, sumatriptan, auranofin, betamethasone, biperiden, ergotamine, estramustine, melphalan, methsuximide, mitotane, norgestrel, phenoxybenzamine, alendronate, amiloride, amlodipine, azathioprine, bromocriptine, chlorpropamide, chlorthalidone, clarithromycin, cortisone, danazol, diflunisal, dipyridamole, estradiol, etoposide, famotidine, fenofibrate, fludrocortsone, isradipine, loperamide, maprotiline, methyltestosterone, nabumetone, nicardipine, nilotinib, nimodipine, nitrofurantoin, nortriptyline, oxcarbazepine, piroxicam, probenecid, propranolol, propylthiouracil, tamoxifen, triazolam, trihexyphenidyl, trimipramine, calcitonin, butoconazole, econazole, amrinone, aloxiprin, aminoglutethimide, astemizole, haloperidol, beclomethasone, bendrofluazide, bexafibrate, bezafibrate, bromazepan, busulphan, camptothecin, carbimazole, cinnarizine, cisapride, clavulanic acid, clioquinol, clofibrate, clotiazepam, cyclizine, darodipine, decoquinate, dexanabinol, dextropropyoxyphene, dicoumarol, dihydrocodeine, domperidone, ethopropazine, fenbufen, fenfluramine, flunarizine, flunitrazepam, fluopromazine, flupenthixol, gliclazide, imidopril, lysuride, mazindol, meclofenamic acid, mefenamic acid, mepenzolate, mesalazine, methaqualone, methoin, methysergide, mianserin, neostigmine, nicoumalone, nitrazepam, norethisterone, oxprenolon, oxyphencylcimine, paramethadione, phenindione, phenylbutazone, pizotifen, probucol, propicillin, pyrantel, sulphadiazine, sulphafurazole, sulphamerazine, sulphapyridine, sulphasalazine, sulphin-pyrazone, sulpiride, terfenadine, zopiclone and zaleplon.

In preferred embodiments, the gastroretentive dosage form comprising the said poorly soluble drug comprises a continuous or discontinuous layer or membrane essentially impervious to the said poorly soluble drug, said membrane further comprising pores, orifices, perforations, openings or like, whereby the said poorly soluble drug is released from the gastroretentive dosage form into the gastric medium.

In further preferred embodiments, the gastroretentive dosage form comprises a reservoir whereto the said poorly soluble drug is confined, said reservoir being bordered by a continuous or discontinuous membrane essentially impervious to the said poorly soluble drug, said membrane further comprising pores, orifices, perforations, openings and the like, whereby the said poorly soluble drug is released from the gastroretentive dosage form into the gastric medium.

The previously described “accordion pill” multilayered configurations (see above mentioned publications) usually comprise an inner layer comprising the drug or diagnostic, a frame layer providing mechanical strength and two outer layers through which the drug diffuses out to the gastric environment. The multilayered structure is compacted or folded into a capsule.

The main advantage of some configurations of these GRDFs is that the gastric retention is achieved through the physical dimensions of the unfolded system augmented by the mechanical strength provided by a separate frame layer. Therefore, the composition for controlled release of a drug has minute influence on the basic performance of the system as whole.

One of the applications of these novel configurations is described in a co-pending PCT application titled “Zaleplon gastroretentive drug delivery system” which is included herein by reference in its entirety. A representative GRDF configuration which is suitable for use with poorly soluble drugs or diagnostics is detailed in Example 1. This configuration exemplifies the lack of dependency of the controlled release mechanism on the gastric retention mechanism and the materials employed therefore.

In a preferred embodiment, there is provided a multilayered gastroretentive dosage form for the delivery of poorly soluble drugs or diagnostics, comprising an inner layer comprising the poorly soluble drug or diagnostic for controlled delivery, a frame layer and two outer layers, wherein at least one of the outer layers has one or more orifices.

In other embodiments the multilayered gastroretentive dosage form optionally comprises a second compartment comprising the poorly soluble drug or diagnostic, wherein said compartment can be present in form of additional layers, denominated “supra-outer” layers, as these are usually present externally to the said outer layers; alternatively the said second compartment can be present as an external coating on the capsule comprising the said multilayered “accordion”. Usually this second compartment provides immediate or close-to-immediate release of the poorly soluble drug or diagnostic.

In some further embodiments, the capsule can be further optionally coated with additional layers, essentially devoid of the drug or diagnostic.

In further embodiment, the external layer has pores, whereas said pores are in either microscopic or macroscopic range. In further preferred embodiments, the pores are mechanical perforations of diameter above 0.1 mm. In other embodiment, the pores are below 0.1 mm and are formed through the manufacturing process of the membrane and not perforated mechanically.

In other embodiments, the pores form spontaneously upon immersion into the gastric medium.

In one embodiment, there is provided a degradable multi-layered gastroretentive dosage form comprising a poorly soluble drug or a diagnostic for the controlled release of said poorly soluble drug or diagnostic in the stomach and gastrointestinal tract of a patient, compacted or folded into a capsule which disintegrates rapidly upon contact with gastric juice and the multilayered structure unfolds rapidly upon contact with gastric juice.

The above gastroretentive dosage form is designed for oral administration and compacted or folded into a standard size capsule which is easily swallowed.

The above multilayered gastroretentive dosage form of poorly soluble drug or diagnostic releases the said poorly soluble drug or diagnostic in a controlled manner regardless of the mechanisms whereby the gastric retention of the system is attained.

In a preferred embodiment, there is provided a degradable, multi-layered gastroretentive dosage form comprising

a. a poorly soluble drug or diagnostic for controlled release incorporated in an internal layer

b. a rigid frame layer and

c. one or two outer layers

d. one or two optional supra-outer layers comprising a second dosage of a poorly soluble drug or diagnostic for immediate release

wherein the above outer layers comprise at least one pore or orifice of discontinuous phase and the said multilayered structure is compacted or folded into a capsule.

In another preferred embodiment, there is provided a degradable, multi-layered gastroretentive dosage form comprising

a. a poorly soluble drug or diagnostic for controlled release incorporated in an internal layer

b. a rigid frame layer and

c. one or two outer layers

wherein the above outer layers comprise at least one pore or orifice of discontinuous phase and the said multilayered structure is compacted or folded into a capsule and the capsule is optionally coated with at least one layer comprising a second dosage of a poorly soluble drug or diagnostic for immediate release and said multilayered structure unfolds rapidly upon contact with gastric juice.

The above structure is compacted or folded into a capsule which disintegrates rapidly upon contact with gastric juice and said structure unfolds rapidly upon contact with gastric juice.

The above structures have preferably two outer layers.

In one embodiment, there is provided a gastroretentive dosage form comprising two outer layers, wherein at least one outer layer has at least one orifice or pore with total area greater than about 0.03 mm², a frame, an inner layer containing a poorly soluble drug or diagnostic for controlled release and wherein the capsule optionally has an immediate release coating

The said gastroretentive dosage form is enveloped, enrobed, coated, sandwiched between or is otherwise contained in an outer layer, wherein said outer layer is permitting passage of gastric media from the environment to the internal layer and inhibiting passage of the poorly soluble drug or diagnostic from the internal layer through the layer to the environment.

The phase continuity in said outer layer is compromised to make at least one orifice or a pore with total area greater than about 0.03 mm² and said orifices or pores enable the release of the said poorly soluble drug or diagnostic into the gastric medium.

In preferred embodiments, the gastroretentive dosage forms of this invention comprise an additional layer covering each outer layer, comprising a powder or a film that prevents adherence of the outer layer onto itself when folded inside the capsule, wherein said layer comprises a powder, a polymer, or a combination thereof. In other preferred embodiments, the external side of the outer layer does not possess self-adhesive properties, or is modified chemically or physically to resist adhesion.

In a preferred embodiment, there is provided a poorly soluble drug or diagnostic gastroretentive dosage form wherein at least one outer layer is perforated in one or more places adjacent to the layer containing the poorly soluble drug or diagnostic producing orifices in order to facilitate the release of the poorly soluble drug or diagnostic from the gastroretentive poorly soluble drug or diagnostic dosage form.

In other preferred embodiments, the orifices are produced prior to assembly of the layered gastroretentive dosage form.

The perforations are carried out using a shaped mechanical blade punch, a laser emitting device or other perforating device known in the art and the diameter of the perforations is between 0.2-2 mm.

In one embodiment, there is provided a gastroretentive dosage form wherein the mean plasma concentration of the poorly soluble drug or diagnostic after single dose administration exhibits a pharmacokinetic profile which is superior to that attainable by a product that is not a gastroretentive and not a controlled release product.

In another embodiment, there is provided a controlled release gastroretentive dosage form wherein the poorly soluble drug or diagnostic gastric retention period is greater than 4 hours or longer than 6-8 hours, up to 12 hours, 16 hours or 24 hours.

In one embodiment, there is provided a method for providing a therapeutic blood plasma concentration of poorly soluble drug or diagnostic over a period of up to 24 hours resulting in improved pharmacological effect or diagnostic result which comprises administering orally to a patient in need thereof a poorly soluble drug or diagnostic multilayered gastroretentive dosage form conducive to pharmacologically effective poorly soluble drug or diagnostic blood plasma levels from about half an hour to about 24 hours.

In another embodiment, there is provided a method for eliminating sharp peaks in the therapeutic blood plasma concentration of poorly soluble drug or diagnostic after administration of poorly soluble drug or diagnostic to a patient in need thereof over a period of up to 24 hours with improved pharmacological ad therapeutic effect which comprises administering orally to a patient in need thereof a poorly soluble drug or diagnostic multilayered gastroretentive dosage form.

In yet another embodiment there is provided a method of treatment of a patient in need thereof, by administering one or more poorly soluble drug or diagnostic gastroretentive dosage forms of this invention.

In one embodiment, there is provided an article of manufacture comprising a number of poorly soluble drug or diagnostic gastroretentive dosage forms of this invention and instructions for their use by swallowing said dosage forms, detailing the number of dosage forms to be used by each population, their dosage and precautions to be taken by the patient.

The outer layers of this invention comprise at least one polymeric combination of a hydrophilic polymer and a polymer insoluble in gastric media, the outer layers being hydratable at a rate greater than the frame layer.

Said polymer in the outer layer insoluble in gastric media is selected from the group consisting of one or more types of polymethacrylate USP.

The outer layers of this invention may comprise propylene glycol as a plasticizer.

The polymeric combination in said outer layers may comprise gelatin.

The amount of gelatin in said outer layers is between about 20% and about 45% of the total outer layer composition.

In one embodiment, the poorly soluble drug or diagnostic gastroretentive dosage form comprises one or more outer layers, preferably two outer layers permitting passage of gastric media from the environment to the internal layer and when intact inhibiting passage of the poorly soluble drug or diagnostic from the internal layer through the layer to the environment.

The rigid frame layer of the GRDF (also referred to as “frame” or “backbone”) provides mechanical strength to the GRDF regardless of the composition of the internal layer.

The polymer in the frame layers of this invention is selected from the group consisting of a degradable enteric polymer which is substantially insoluble at pH less than 5.5 and a plasticizer.

The enteric polymer in the frame layer is selected from the group consisting of cellulose acetate phthalate, hypromelose phthalate, hydroxypropyl methylcellulose acetate succinate, polyvinyl acetate phthalate and methylmethacrylate-methacrylic acid copolymers.

The polymer in the frame layer may be a polymethacrylate copolymer.

The frame layer in the gastroretentive dosage forms of this invention has a mechanical strength described with Young's modulus ranging from about 0.5 to 15 Kgf/mm² and stress of about 0.03 to about 0.6 Kgf/mm² after 1 hour in simulated gastric fluid.

The composition of said frame layer optionally further comprises a filler, a surface-active agent, an additional plasticizer and other materials suitable for such composition.

The inner layer of the GRDFs of this invention comprises a poorly soluble drug or diagnostic and at least one polymer, whereas the poorly soluble drug or diagnostic is substantially uniformly dispersed in the polymer or forms a solid solution therewith and wherefrom the poorly soluble drug or diagnostic is released upon subjecting the gastroretentive dosage form into a gastric medium.

The above at least one polymer is chosen from water-soluble polymers.

In some preferred embodiments, said polymer is soluble in an organic solvent as well

In other preferred embodiments said polymer is capable of increasing the stability of the poorly soluble drug or diagnostic and/or their solubility in aqueous media.

The inner layer may further comprise at least one plasticizer.

The inner layer composition of the GRDFs of this invention may optionally further comprise a filler, a surface-active agent, and/or other materials suitable for such composition.

The poorly soluble drug or diagnostic present in the inner layer may be in the form of a powder, granulated powder, miniature tablets, coated powder, semisolid composition or any other form known to the versed in the art.

The layers of the GRDFs of this invention may be joined together with ultrasonic welding.

Alternatively, the layers may be joined together with a glue or by the means of a suitable solvent.

In one embodiment, there is provided a gastroretentive dosage form wherein the multilayered composition unfolds to a length of at least 20 mm within 15 minutes of being exposed to gastric fluid.

In another embodiment, the gastroretentive drug formulation is fully degradable within 3 hours in simulated intestinal fluid.

In the current embodiments, the gastroretentive dosage form of this invention includes a frame layer, an internal layer and two outer layers. The outer layers are two films which are slightly larger than the frame layer and which are sealed or welded together around their perimeter and completely envelop the frame and the internal layer. Along with welding which connects the outer layers together, the outer portion of the frame layer is also welded to the outer layers. The compositions, ingredients and structure of the various layers forming the GRDF are detailed in the following. Illustrative examples of the formulations out of which the layers are prepared are provided in the Examples.

In some embodiments, the gastroretentive dosage form may comprise an additional dose of poorly soluble drug or diagnostic contained in a coating applied onto the capsule of the gastroretentive dosage form, and said coating comprises at least one layer.

The said coating comprises at least one polymer, whereof the at least one polymer is preferably instantly soluble in gastric medium.

The poorly soluble drug or diagnostic in the above coating is uniformly dispersed or dissolved in said coating.

The coating may further comprise a plasticizer or a combination of plasticizers.

The coating may further optionally comprise at least one anti-tacking agent or another filler.

In one embodiment, the gastroretentive dosage form may optionally comprise an additional layer covering each outer layer or each supra-outer layer, comprising a powder or a film that prevents adherence of the outer membrane onto itself when folded inside the capsule, wherein said layer comprises a powder, a polymer, or a combination thereof.

Some of the GRDFs of this invention have in addition to the inner layer, frame layer and outer layer or layers, one or two additional external layers, named “supra-outer layers”.

In another embodiment, the one or two supra-outer layers are affixed to the outer layer on one or two sides of the gastroretentive dosage form, and wherein these supra-outer layers comprise a poorly soluble drug or diagnostic and one or more inactive ingredients selected from the group consisting of polymers, preferably water soluble polymers, a plasticizer, a solubilizing agent intended for immediate release of the drug in the stomach, a disintegrant and a glidant, or any combination of ingredients capable of performing one or more of the said functions.

In preferred embodiments, the perforations in the outer membrane are formed with a suitable mechanical tooling, such as a shaped blade or needle, or alternative methods, such as laser cutting or burning and the like.

It is appreciated that certain features of the invention which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent and patent application was specifically and individually indicated to be incorporated herein by reference. In addition, citation or identification of any reference in this application shall not be construed as an admission that such reference is available as prior art to the present invention.

The detailed description of the present invention is further illustrated by the following examples, which are illustrative only and are not to be construed as limiting the scope of the invention. Variations and equivalents of these examples will be apparent to those skilled in the art in light of the present disclosure, the drawings and the claims herein.

Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### EXAMPLES

The following examples further illustrate the invention but, of course, should not be construed as in any way limiting its scope.

### Example 1

### GRDF Type: IR Capsule Coating, Outer-Frame-Inner-Outer Layer (Each Outer has 4 Orifices)

TABLE 1

| IR Coating | (mg per capsule) |
|---|---|
| Triethyl Citrate (TEC) | 2.5 |
| PEG 400 | 1.25 |
| Eudragit E PO | 12.5 |
| Talc Extra fine | 2.5 |
| Poorly soluble drug or diagnostic | According to case |

TABLE 2

| Outer Layer | mg |
|---|---|
| Potassium hydroxide | 4.1 |
| Propylene glycol | 63.9 |
| Gelatin (Fish) | 63.9 |
| Eudragit L100-55 | 16 |
| Eudragit L100 | 16 |
| Eudragit S100 | 32 |

TABLE 3

| Rigid Frame Layer | mg |
|---|---|
| Lutrol F127 | 50 |
| Eudragit L100-55 | 29.2 |
| Eudragit L100 | 117.1 |
| Lactose | 62.3 |
| Talc | 15.6 |

TABLE 4

| Inner Layer | mg |
|---|---|
| PEG 400 | 7.5 |
| Kollidon 90 F | 30.0 |
| Methocel E 3 | 10.0 |
| Poorly soluble drug or diagnostic | According to case |

### Example 2

### GRDF Type: IR—Capsule Coating, Outer-Frame-Inner-Outer Layer (Each Outer Layer has 2 Orifices)

TABLE 5

| IR Coating | (mg per capsule) |
|---|---|
| Triethyl Citrate (TEC) | 2.0 |
| PEG 400 | 1.0 |
| Eudragit E PO | 10.0 |
| Talc Extra fine | 2.0 |

TABLE 6

| Outer Layer | mg |
|---|---|
| Potassium hydroxide | 4.1 |
| Propylene glycol | 63.9 |
| Gelatin (Fish) | 63.9 |
| Eudragit L100-55 | 16 |
| Eudragit L100 | 16 |
| Eudragit S100 | 32 |

TABLE 7

| Rigid Frame Layer | mg |
|---|---|
| Lutrol F127 | 50 |
| Eudragit L100-55 | 29.2 |
| Eudragit L100 | 117.1 |
| Lactose | 62.3 |
| Talc | 15.6 |

TABLE 8

| Inner Layer | mg |
|---|---|
| PEG 400 | 11.25 |
| Kollidon 90 F | 45.0 |
| Methocel E 3 | 15.0 |
| Poorly soluble drug or diagnostic | According to case |

### Example 3

### GRDF Type: IR—Two Supra-Outer Films, Outer-Frame-Inner-Outer (Each Outer Layer has 4 Orifices)

TABLE 9

| Outer Layer | mg |
|---|---|
| Potassium hydroxide | 4.1 |
| Propylene glycol | 63.9 |
| Gelatin (Fish) | 63.9 |
| Eudragit L100-55 | 16 |
| Eudragit L100 | 16 |
| Eudragit S100 | 32 |

TABLE 10

| Inner Layer | Amount/GRDF (mg) |
|---|---|
| Poorly soluble drug or diagnostic | According to case |
| Klucel EF (Hydroxypropylcellulose) | 7.5 |
| Klucel GF (Hydroxypropylcellulose) | 7.5 |
| Kollidon K30 (PVP, Povidone) | 3.0 |
| PEG 400 (Polyethyleneglycol) | 1.5 |

TABLE 11

| Rigid Frame Layer | Amount/GRDF (mg) |
|---|---|
| Lutrol F127 (poloxamer 407) | 92.5 |
| Eudragit L100 | 212.7 |
| Eudragit L100-55 | 53.2 |
| Lactose | 113.3 |
| Talc | 28.2 |

TABLE 12

| Two Supra-outer Layers | Amount/GRDF (mg) |
|---|---|
| Poorly soluble drug or diagnostic | According to case |
| Methylcellulose | 18.0 |
| Kollidon K30 (PVP, Povidon) | 30.0 |
| SLS (SDS, Sodium Laurylsulfate) | 15.0 |
| PEG 400 (Polyethyleneglycol) | 8.0 |

1-40. (canceled)

41. A degradable gastroretentive dosage form comprising a multilayered structure comprising a poorly soluble drug or a diagnostic for controlled release in the stomach and the upper parts of gastrointestinal tract of a subject; wherein said multi-layered structure is compacted or folded into a capsule, optionally a standard size capsule, which capsule disintegrates rapidly upon contact with gastric juice; wherein said multilayered structure unfolds rapidly upon contact with gastric juice; and wherein the gastric retention is optionally attained independently of materials providing for the said controlled release of said poorly soluble drug or diagnostic.

42. The degradable, gastroretentive dosage form of claim 41, wherein said multilayered structure comprises
a. an internal layer comprising a first dose of a poorly soluble drug or diagnostic for controlled release;
b. a rigid frame layer;
c. one or two outer layers;
wherein at least one of said one or two outer layers comprises at least one pore or orifice of discontinuous phase; and wherein said gastroretentive dosage form optionally further comprises a second dose of said poorly soluble drug or diagnostic for immediate release, wherein said second dose is provided by at least one of:
d. said capsule further comprising at least one coating comprising said second dose; and
e. said multilayered structure further comprising one or two supra-outer layers comprising said second dose.

43. The gastroretentive dosage form of claim 42, wherein said multilayered structure has two outer layers, and optionally wherein at least one of said two outer layers has at least one orifice or pore with area greater than about 0.03 mm2.

44. The gastroretentive dosage form of claim 43 wherein at least one of said two outer layers permits passage of gastric media from the environment to the internal layer and inhibits passage of the poorly soluble drug or diagnostic from the internal layer through the layer to the environment, and wherein said at least one orifice or pore enables the release of said poorly soluble drug or diagnostic into the gastric medium.

45. The gastroretentive dosage form of claim 42, further comprising an additional layer covering each of said outer layers or each of said supra-outer layers, where present, comprising a powder or a film that prevents adherence of the outer or supra-outer layer onto itself when folded inside the capsule, wherein said layer comprises a powder, a polymer, or a combination thereof.

46. The gastroretentive dosage form of claim 42, wherein said poorly soluble drug or diagnostic is a very slightly soluble material as defined by the United States Pharmacopeia 31, or with the aqueous solubility below this definition.

47. The gastroretentive poorly soluble drug or diagnostic dosage forms of claim 43, wherein the perforations are made using a shaped mechanical blade punch, a laser emitting device or other perforating device and wherein the diameter of the perforations is between 0.2-2 mm.

48. The gastroretentive dosage form of claim 41, wherein said poorly soluble drug or diagnostic is retained in the stomach for gastric retention period that is greater than 4 hours or longer than 6-8 hours, up to 12 hours, 16 hours or 24 hours.

49. The gastroretentive dosage form of claim 42, wherein each said one or two outer layers comprises a polymeric combination of a hydrophilic polymer and a polymer insoluble in gastric media, wherein each said one or two outer layers is hydratable at a rate greater than that of the said rigid frame layer, and optionally wherein said polymer insoluble in gastric media is selected from one or more types of polymethacrylate USP, and optionally wherein said one or two outer layers comprise propylene glycol as a plasticizer.

50. The gastroretentive dosage form of claim 49, wherein the polymeric combination in said one or two outer layers comprises gelatin, optionally at an amount of between about 20% and about 45% of the total composition of said one or two outer layers.

51. The gastroretentive dosage form of claim 42, wherein the polymer in the rigid frame layer is selected from a degradable enteric polymer which is substantially insoluble at pH less than 5.5, and wherein said rigid frame layer further comprises a plasticizer, wherein said enteric polymer in said rigid frame layer is optionally selected from the group consisting of cellulose acetate phthalate, hypromellose phthalate, hydroxypropyl methylcellulose acetate succinate, polyvinyl acetate phthalate and methylmethacrylate-methacrylic acid copolymers.

52. The gastroretentive dosage form of claim 51, wherein said enteric polymer in said rigid frame layer is polymethacrylate copolymer USP.

53. The gastroretentive dosage form of claim 51, wherein said rigid frame layer has a mechanical strength described with Young's modulus ranging from about 0.5 to 15 Kgf/mm and stress of about 0.03 to about 0.6 Kgf/mm after 1 hour in simulated gastric fluid.

54. The gastroretentive dosage form of claim 51 wherein said rigid frame layer further comprises at least one of a filler, a surface-active agent and an additional plasticizer.

55. The gastroretentive dosage form of claim 42, wherein said internal layer further comprises at least one polymer in which said poorly soluble drug or diagnostic is substantially uniformly dispersed or with which of forms a solid solution, and wherein said poorly soluble drug or diagnostic is released from said polymer or said solid solution upon subjecting the gastroretentive dosage form into a gastric medium, wherein said at least one polymer is optionally a water-soluble polymer, and wherein said poorly soluble drug or diagnostic is optionally in form of a powder, a granulated powder, a miniature tablets, a coated powder or a semisolid composition.

56. The gastroretentive dosage form of claim 55, wherein said internal layer further comprises at least one plasticizer, and inner layer composition optionally further comprises at least on of a filler and a surface-active agent.

57. The gastroretentive dosage form of claim 42, wherein the multilayered structure is joined together by ultrasonic welding, or with a glue or by means of a suitable solvent.

58. The gastroretentive dosage form of claim 57, wherein said multilayered structure unfolds upon contact with gastric juice to a length of at least 20 mm within 15 minutes of being exposed to the gastric fluid, and wherein said multilayered structure is optionally fully degradable within 3 hours from being placed in simulated intestinal fluid.

59. The gastroretentive dosage form of claim 41, wherein said poorly soluble drug or diagnostic is selected from the group consisting of progesterone, tacrolimus, estradiol, budesonide, dipyridamole, norgestrel, alendronate, amlodipine, sumatriptan, auranofin, betamethasone, biperiden, ergotamine, estramustine, melphalan, methsuximide, mitotane, norgestrel, phenoxy-benzamine, alendronate, amiloride, amlodipine, azathioprine, bromocriptine, chlorpropamide, chlorthalidone, clarithromycin, cortisone, danazol, diflunisal, dipyridamole, estradiol, etoposide, famotidine, fenofibrate, fludrocortsone, isradipine, loperamide, maprotiline, methyltestosterone, nabumetone, nicardipine, nilotinib, nimodipine, nitrofurantoin, nortriptyline, oxcarbazepine, piroxicam, probenecid, propranolol, propylthiouracil, tamoxifen, triazolam, trihexyphenidyl, trimipramine, calcitonin, butoconazole, econazole, amrinone, aloxiprin, aminoglutethimide, astemizole, haloperidol, beclomethasone, bendrofluazide, bexafibrate, bezafibrate, bromazepan, busulphan, camptothecin, carbimazole, cinnarizine, cisapride, clavulanic, clioquinol, clofibrate, clotiazepam, cyclizine, darodipine, decoquinate, dexanabinol, dextropropyoxyphene, dicoumarol, dihydrocodeine, domperidone, ethopropazine, fenbufen, fenfluramine, flunarizine, flunitrazepam, fluopromazine, flupenthixol, gliclazide, imidopril, lysuride, mazindol, meclofenamic, mefenamic, mepenzolate, mesalazine, methaqualone, methoin, methysergide, mianserin, neostigmine, nicoumalone, nitrazepam, norethisterone, oxprenolon, oxyphencylcimine, paramethadione, phenindione, phenylbutazone, pizotifen, probucol, propicillin, pyrantel, sulphadiazine, sulphafurazole, sulphamerazine, sulphapyridine, sulphasalazine, sulphin-pyrazone, sulpiride, terfenadine, zopiclone and zaleplon.

60. A method for providing a therapeutic blood plasma concentration of poorly soluble drug or diagnostic over a period of up to 24 hours, comprising administering orally to a patient in need thereof a gastroretentive dosage form of claim 41, said administration resulting in improved pharmacological effect or improved accuracy of diagnostic result, wherein said improved pharmacological effect or improved accuracy of diagnostic result is produced by maintaining pharmacologically or diagnostically effective blood plasma levels of said poorly soluble drug or diagnostic for from about half an hour to about 24 hours, optionally without sharp peaks in said blood plasma levels of said poorly soluble drug or diagnostic.

61. A method of treatment of a patient in need thereof, comprising administering orally to said patient one or more poorly soluble drug or diagnostic gastroretentive dosage forms of claim 41, wherein the condition of said patient is responsive to said poorly soluble drug or diagnostic.
